(19) United States
(12) Patent Application Publication
(10) Pub. No.: US 2014/0100646 A1
(43) Pub. Date: Apr. 10, 2014

(54) METHOD AND DEVICES FOR FLOW OCCLUSION DURING DEVICE EXCHANGES

(71) Applicant: AccessClosure, Inc., Mountain View, CA (US)

(72) Inventors: Ali H.M. Hassan, Palo Alto, CA (US); Ronald R. Hundertmark, San Mateo, CA (US); Kevin To, San Jose, CA (US); Kristin N. Meader, San Francisco, CA (US); Todd C. Bitner, Waltham, MA (US)

(73) Assignee: AccessClosure, Inc., Mountain View, CA (US)

(21) Appl. No.: 14/047,943

(22) Filed: Oct. 7, 2013

Related U.S. Application Data
(60) Provisional application No. 61/711,368, filed on Oct. 9, 2012.

(51) Int. Cl. A61F 2/958

(52) U.S. Cl. CPC A61F 2/958
USPC 623/1.11

(57) ABSTRACT

A method of treating an injured blood vessel of a patient may first involve inflating a balloon of an access wire balloon catheter within the injured blood vessel to reduce blood flow past an injury site in the vessel. After inflation, the method may involve attaching an extension wire to an extra-corporeal end of the access wire balloon catheter that resides outside the patient. When the extension wire is attached, an inflation port of the access wire device is disposed outside the patient and between a free end of the extension wire and the balloon of the access wire balloon catheter. The method may further include advancing at least a first treatment catheter into the blood vessel over the access wire balloon catheter and at least a portion of the extension wire and treating the injured blood vessel using the first treatment catheter.

### CROSS-REFERENCE TO RELATED APPLICATIONS

The present application claims priority benefit under 35 U.S.C. §119(e) of U.S. Provisional Application Ser. No. 61/711,368, filed Oct. 9, 2012, entitled “Method and Devices for Flow Occlusion During Device Exchanges,” the disclosure of which is incorporated herein by reference in its entirety.

### INCORPORATION BY REFERENCE

This application incorporates by reference U.S. patent application Ser. No. 13/531,227, filed Jun. 22, 2012, and entitled “Method and Devices for Flow Occlusion During Device Exchanges.”

### BACKGROUND

1. Field

The field of the present application pertains to medical devices, and more particularly, to methods and systems for maintaining vascular access and/or minimizing bleeding, for example, during and after catheter-based interventions, for example, in the settings of device exchanges, vascular access closure, and the management of vascular complications.

2. Description of the Related Art

Catheter-based medical procedures using large diameter (or “large bore”) vascular access sheaths are becoming increasingly more common. Two examples of such large bore catheterization procedures that are gaining rapid popularity are Transcatheter Aortic Valve Implantation (“TAVI”) and Endovascular abdominal Aortic aneurysm Repair (“EVAR”). Although these procedures may often be effective at treating the condition addressed, they often cause injury to the blood vessel in which the large bore vascular access catheter is inserted to gain access for performing the procedure. In fact, vascular injury requiring treatment occurs in as many as 30-40% of large bore vascular procedures, according to some sources. Injury to the blood vessel may include perforation, rupture and/or dissection, which causes blood to flow out of the artery (“extravascular bleeding”), often requiring emergency surgery to repair the damaged blood vessel wall. If not properly treated, such a vascular injury may lead to anemia, hypotension, or even death.

Vascular injury during large bore intravascular procedures is typically caused by the vascular access sheath itself and/or one or more instruments passed through the sheath to perform the procedure. Larger diameter vascular access sheaths are required in a number of catheter-based procedures, such as those mentioned above, where relatively large catheters/instruments must be passed through the sheath. Several other factors may increase the risk of vascular injury, including occlusive disease of the access vessel(s) and tortuosity/angulation of the access vessel(s). Another vascular injury caused by large bore intravascular procedures that can be challenging is the access site itself. Typically, large bore catheterizations create a significantly large arteriotomy, due to a disproportionately large ratio of the diameter of the vascular access catheter to the diameter of the artery in which it is placed. Large arteriotomies may require special management and multiple steps during closure. This may lead to significant blood loss while access closure is attempted.

Several techniques have been attempted to reduce the incidence of vascular injury in large bore vascular access procedures. For example, preoperative imaging of the blood vessel to be accessed, in the form of CT and MR angiography, may provide the physician with an idea of the anatomy of the vessel. If a particular vessel appears on imaging studies to be relatively tortuous or small, possible adjunctive maneuvers to prevent arterial dissection include pre-dilatation angioplasty of the iliofemoral vessels prior to large bore sheath placement, utilization of smaller access sheaths when possible, stiffer wires to aid in sheath placement/withdrawal and/or use of hydrophobic or expandable sheaths. In another attempt at preventing vessel injury, sheath placement may be performed under fluoroscopic guidance, and advancement may be halted when resistance is encountered. Despite the availability of these techniques, vascular injury requiring treatment still occurs in a large percentage of large bore vascular procedures.

Vascular injuries caused by intravascular procedures are generally quite difficult to diagnose and treat. When an arterial dissection occurs, it often remains undetected until the catheterization procedure is completed and the vascular access sheath is removed. For example, upon removal of the access sheath, large segments of the dissected vessel wall may be released within the vessel. The dissected vessel wall may lead to a breach in the artery wall, a flow-limiting stenosis, or distal embolization. Perforation or rupture of the iliofemoral artery segment may occur from persistent attempts to place large access sheaths in iliac arteries that are too small, too diseased, and/or too tortuous. Here too, a perforation may be likely to remain silent until sheath withdrawal.

Generally, vascular perforations and dissections caused by large bore vascular procedures allow very little time for the interventionalist to react. Frequently, these vascular injuries are associated with serious clinical sequelae, such as massive internal (retroperitoneal) bleeding, abrupt vessel closure, vital organ injuries, and emergency surgeries. In some cases, an interventionalist may first attempt to repair a vascular injury using an endovascular approach. First, the injury site may be controlled/stabilized with a balloon catheter, in an attempt to seal off the breached vessel wall and/or regain hemodynamic stability in the presence of appropriate resuscitation and transfusion of the patient by the anesthesiologist. Subsequently, endovascular treatment solutions may be attempted, for example if wire access is maintained through the true lumen. This may involve placement of one or more balloons, stents, or covered stents across the dissection/perforation. If the hemorrhage is controlled with these maneuvers and the patient is hemodynamically stabilized, significant reduction in morbidity and mortality may be realized. If attempts at endovascular repair of the vessel fail, emergency surgery is typically performed.

Presently, vascular injuries and complications occurring during and after large bore intravascular procedures are managed using a contralateral balloon occlusion technique (“CBOT”). CBOT involves accessing the contralateral femoral artery (the femoral artery opposite the one in which the large bore vascular access sheath is placed) with a separate access sheath, and then advancing and maneuvering a series of different guidewires, sheaths and catheters into the injured (ipsilateral) femoral or iliofemoral artery to treat the injury. Eventually, a (pre-sized) standard balloon catheter is advanced into the injured artery, and the balloon is inflated to reduce blood flow into the area of injury, thus stabilizing the injury until a repair procedure can be performed. Typically, CBOT involves at least the following steps: (1) Place a catheter within the contralateral iliofemoral artery (this catheter may already be in place for use in injecting contrast during the intravascular procedure); (2) Advance a thin, hydrophilic guidewire through the catheter and into the vascular access sheath located in the ipsilateral iliofemoral artery; (3) Remove the first catheter from the contralateral iliofemoral artery; (4) Advance a second, longer catheter over the guidewire and into the vascular access sheath; (5) Remove the thin, hydrophilic guidewire; (6) Advance a second, stiffer guidewire through the catheter into the vascular access sheath; (7) In some cases, an addition step at this point may involve increasing the size of the arteriotomy on the contralateral side to accommodate one or more balloon catheter and/or treatment devices for treating arterial trauma on the ipsilateral side; (8) Advance a balloon catheter over the stiffer guidewire into the damaged artery; (9) Inflate the balloon on the catheter to occlude the artery; (10) Advance one or more treatment devices, such as a stent delivery device, to the site of injury and repair the injury.

As this description suggests, the current CBOT technique requires many steps and exchanges of guidewire and catheters, most of which need to be carefully guided into a vascular access catheter in the opposite (ipsilateral) iliofemoral artery. Thus, the procedure is quite challenging and cumbersome. Although considered the standard of care in the management of vascular complications, the CBOT technique may not provide immediate stabilization of an injured segment, may lack ipsilateral device control, and/or may not provide ready access for additional therapeutics such as stents, other balloons, and the like.

Various embodiments developed to address the above concerns are described in U.S. patent application Ser. No. 13/531,227, which was previously incorporated by reference. A number of alternative embodiments are described herein.

### SUMMARY

Certain aspects of this disclosure are directed toward a method of treating an injured blood vessel of a patient. The method can include inflating a balloon of an access wire balloon catheter within the injured blood vessel to reduce blood flow past an injury site in the vessel, and attaching an extension wire to an extra-corporeal end of the access wire balloon catheter that resides outside the patient. When the extension wire is attached, an inflation port of the access wire device can be disposed outside the patient and between a free end of the extension wire and the balloon of the access wire balloon catheter. The method can also include advancing at least a first treatment catheter into the blood vessel over the access wire balloon catheter and at least a portion of the extension wire, and treating the injured blood vessel using the first treatment catheter.

The above-mentioned method can also include removing the first treatment catheter from the blood vessel over the access wire balloon catheter and at least a portion of the extension wire, advancing a second treatment catheter into the blood vessel over the access wire balloon catheter and at least a portion of the extension wire, and further treating the injured blood vessel using the second treatment catheter.

Any of the above-mentioned methods can include deflating the balloon and removing the access wire balloon catheter from the blood vessel while the extension wire is still attached.

Any of the above-mentioned methods can include, before the inflating step, detecting an injury in the injured blood vessel, and positioning the balloon of the access wire balloon catheter device in a desired location in the blood vessel to provide at least partial occlusion of the vessel after inflation of the balloon.

In any of the above-mentioned methods, inflating the balloon can include inflating at a location of the vascular injury.

In any of the above-mentioned methods, inflating the balloon can include inflating at a location upstream of the vascular injury.

In any of the above-mentioned methods, the first treatment catheter can include a stent deployment catheter. Treating the injury can include placing a stent in the blood vessel.

Certain aspects of this disclosure are directed toward a system for facilitating treatment of an injured blood vessel of a patient. The system can include an access wire balloon catheter. The balloon catheter can include an elongate tubular body with a proximal end, a distal end, and a lumen extending longitudinally through at least part of the body. The balloon catheter can also include an inflatable balloon disposed on the elongate body closer to the distal end than to the proximal end and in communication with the lumen, and a valve at or near the proximal end of the elongate body configured to couple with an inflation device to allow for inflation and deflation of the balloon. The system can include a first coupling member at the proximal end, and an extension wire having a second coupling member at one end. The first and second coupling members can be configured to attach to one another to connect the proximal end of the access wire balloon catheter with one end of the extension wire. An outer diameter of the access wire balloon catheter can be approximately the same as an outer diameter of the extension wire, at least in an area around a connection between the access wire balloon catheter and the extension wire.

In the above-mentioned system, the first and second coupling members can attach to one another via a mechanism selected from the group consisting of threads, crimping, friction fit, shaped fit, phase change material(s), ball and socket fit, hook and pin, magnetics, and interference fit.

In any of the above-mentioned systems, the access wire balloon catheter can have a length of between about 85 cm and about 150 cm. A total length of the combined access wire balloon catheter and extension wire can be between about 200 cm and about 350 cm.

In any of the above-mentioned systems, when the extension wire is connected to the access wire balloon catheter, the valve can reside between a connection of the first and second connection members and the balloon of the access wire balloon catheter.

Certain aspects of this disclosure are directed toward a device for facilitating treatment of an injured blood vessel of a patient. The device can include an extension wire having a coupling member at one end for coupling with a corresponding coupling member on an access wire balloon catheter device used to occlude blood flow in the injured blood vessel. An outer diameter of the extension wire can be approximately the same as an outer diameter of the access wire balloon catheter, at least in an area around a connection between the extension wire and the access wire balloon catheter.

In the above-mentioned device, the coupling member can couple with the corresponding coupling member via a mechanism selected from the group consisting of threads, crimping, friction fit, shaped fit, phase change material(s), ball and socket fit, hook and pin, and interference fit.

In any of the above-mentioned devices, the extension wire can have a length of between about 100 cm and about 215 cm.

In any of the above-mentioned devices, the extension wire can connect to one end of the access wire balloon catheter such that a valve of the access wire balloon catheter can be distal to the connection.

Any feature, structure, or step disclosed herein can be replaced with or combined with any other feature, structure, or step disclosed herein, or omitted. Further, for purposes of summarizing the disclosure, certain aspects, advantages, and features of the inventions have been described herein. It is to be understood that not necessarily any or all such advantages are achieved in accordance with any particular embodiment of the inventions disclosed herein. No aspects of this disclosure are essential or indispensable.

### BRIEF DESCRIPTION OF THE DRAWINGS

FIGS. 1A and 1B are side, cross-sectional views of one end of an access wire balloon and a mating end of an extension wire for extending the length of the access wire balloon, where the two ends are connected via a threaded insert, according to one embodiment;

FIGS. 2A and 2B are side, cross-sectional views of one end of an access wire balloon and a mating end of an extension wire for extending the length of the access wire balloon, where the two ends are connected via crimping, according to another embodiment;

FIG. 3 is a side, cross-sectional view of one end of an access wire balloon and a mating end of an extension wire for extending the length of the access wire balloon, where the two ends are connected via a friction fit, according to another embodiment;

FIGS. 4A and 4B are side, cross-sectional views of one end of an access wire balloon and a mating end of an extension wire for extending the length of the access wire balloon, where the two ends are connected via an arrow-head shaped protrusion, according to another embodiment;

FIGS. 5A-5C are side, cross-sectional views of one end of an access wire balloon and a mating end of an extension wire for extending the length of the access wire balloon, where the two ends are connected via an insert that undergoes a phase change, according to another embodiment;

FIGS. 6A-6C are side, cross-sectional views of one end of an access wire balloon and a mating end of an extension wire for extending the length of the access wire balloon, where the two ends are connected via a shaped end of the extension wire, according to another embodiment;

FIGS. 7A and 7B are side, cross-sectional views of one end of an access wire balloon and a mating end of an extension wire for extending the length of the access wire balloon, where the two ends are connected via a hook and pin mechanism, according to another embodiment;

FIGS. 8A and 8B are side, cross-sectional views of one end of an access wire balloon and a mating end of an extension wire for extending the length of the access wire balloon, where the two ends are connected via an interference fit, according to another embodiment;

FIGS. 9A-9I are diagrammatic illustrations of a femoral artery, iliofemoral segment, and aorta portion, showing an exemplary method for stabilizing vascular injuries and managing blood flow during interventions to treat vascular injuries;

FIG. 10 is a perspective view of a guide wire balloon system, including close-up views of an inflation device, a balloon section of a guide wire device, and a core wire and distal tip of the guide wire device according to one embodiment; and

FIG. 11 is a side, cross-sectional view of a balloon section of a guide wire device.

Various embodiments are depicted in the accompanying drawings for illustrative purposes, and should in no way be interpreted as limiting the scope of the embodiments. Furthermore, various features of different disclosed embodiments can be combined to form additional embodiments, which are part of this disclosure.

### DETAILED DESCRIPTION

Although certain embodiments and examples are disclosed below, inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

Various embodiments of an access wire balloon catheter are described in U.S. patent application Ser. No. 13/531,227, which was previously incorporated by reference. Generally, access wire balloon catheter includes a shaft with a central inflation lumen that communicates with a flow regulator. Typically, the flow regulator is located at one end of the access wire catheter that remains outside of a patient during a procedure (i.e., the “extra-corporeal tip” of the access wire catheter). During catheterization of the iliofemoral artery, the access wire balloon catheter (also called the “primary catheter”) allows for introduction and removal (i.e., “exchange”) of one or more additional catheters/repair devices (also called “secondary catheters”) into the artery while providing occlusion of blood flow. The secondary catheters are passed in and out of the artery over the access wire balloon catheter.

During exchange of the secondary device(s), the primary balloon catheter must allow for co-axial (over-the-wire) insertion, while providing flow occlusion. Given that an exchange length of the primary balloon catheter is required in order to enable the exchange of the secondary device, the working length of the primary balloon catheter (i.e., the access wire balloon catheter) should usually be at least about 200 cm and more preferably at least about 260 cm. Typically, the total ideal length of the access wire balloon catheter is about 260-350 cm. Making an access wire balloon of this length, however, has a number of technical challenges. For example, extending the central lumen of the access wire balloon catheter for the purpose of providing an adequate length for secondary catheter exchanges (i.e., at least about 200 cm and ideally at least about 260 cm) could be associated with long balloon inflation/deflation times, extended length that is vulnerable to kinks, bends impacting balloon inflation performance, and high costs of manufacturing. Additionally, during the initial stages of catheterization (i.e., prior to the secondary device exchange), it is easier to use and manipulate a primary access wire balloon catheter of shorter length, such as less than about 260 cm, and ideally less than about 200 cm. Even smaller lengths for the access wire balloon catheter, such as less than about 150 cm or less than about 100 cm, would be even more advantageous during the initial stages of catheterization and positioning, before using secondary catheters. Therefore, it would be advantageous to provide an access wire balloon catheter with an extendable working length.

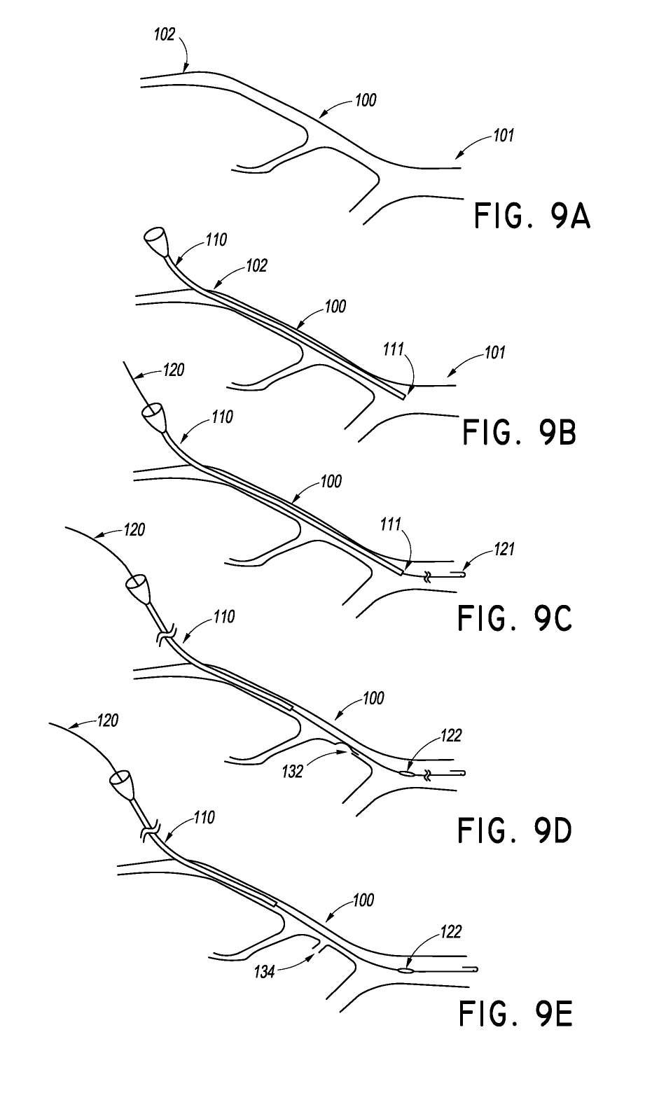

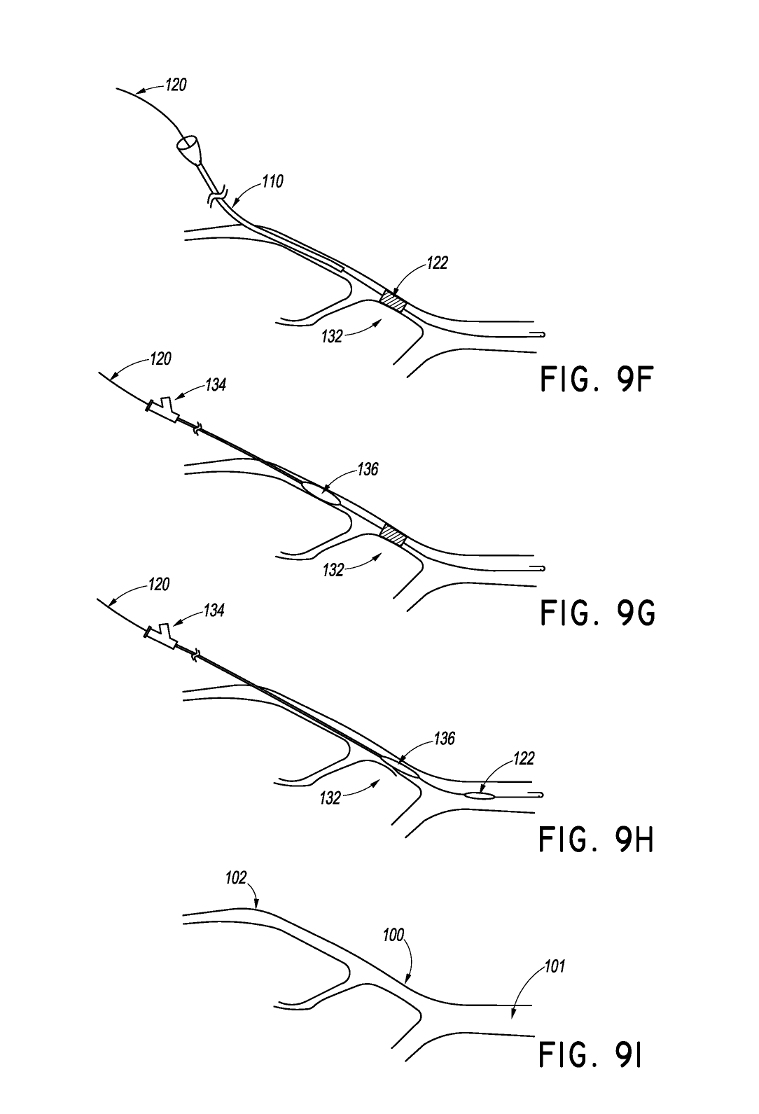

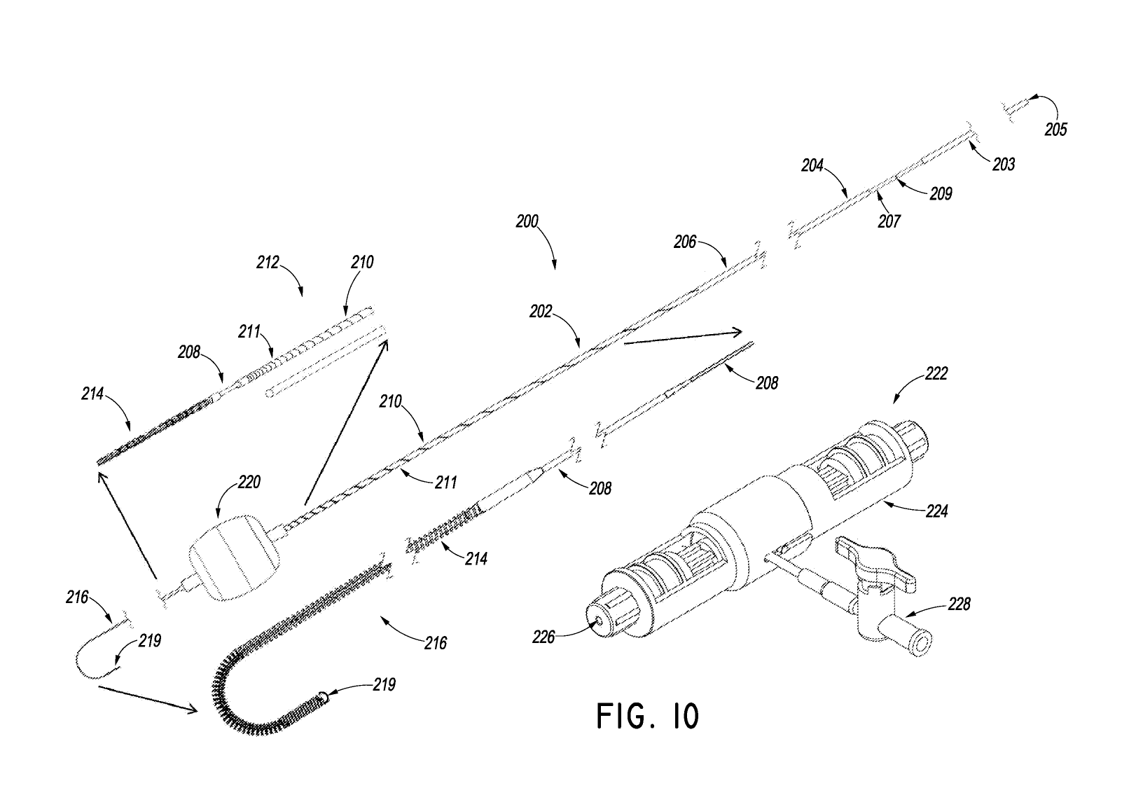

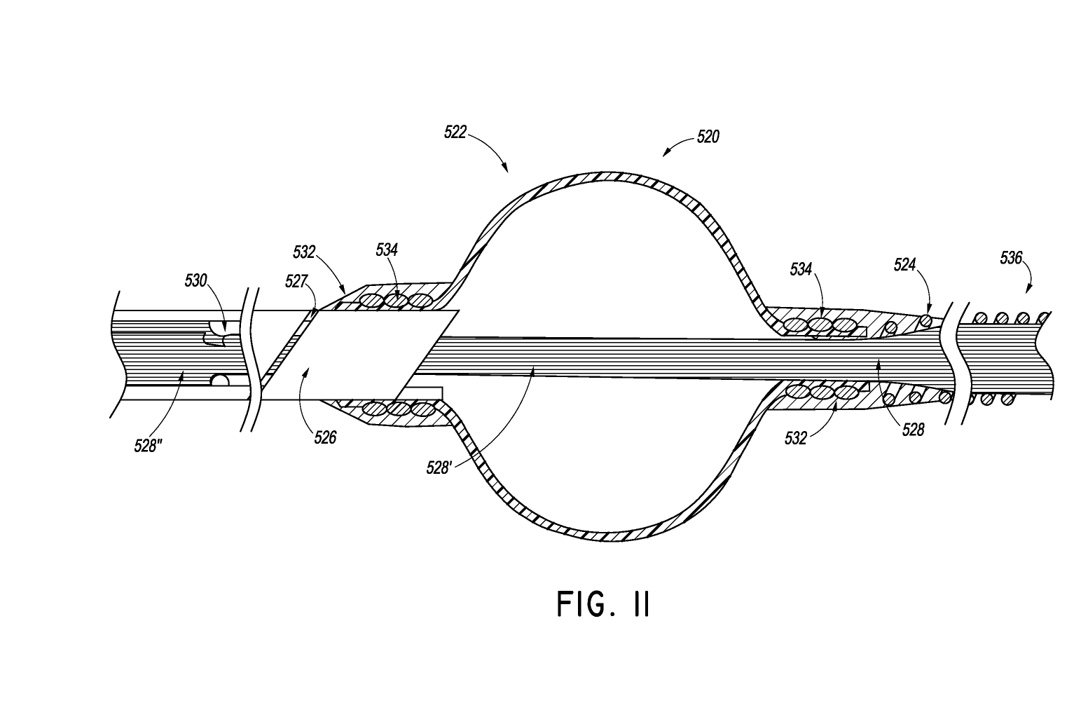

Referring now to FIGS. 9A-9I, a method is provided for managing vascular complications and/or controlling bleeding during or after trans-femoral catheterization. FIG. 9A illustrates the femoral artery 102, iliofemoral artery 100 (or “iliofemoral segment”) and a small portion of the aorta 101. As shown in FIG. 9B, the method may initially include inserting a vascular access sheath 110 (or “procedure sheath”) into the femoral artery 102 and advancing its distal end 111 into the iliofemoral segment 100 for conducting a catheterization procedure, similar to the previous embodiment. In most embodiments, the vascular access sheath 110 will be used for performing one or more intravascular or transvascular procedures, such as but not limited to EVAR or TAVI (also called transvascular aortic valve replacement, or “TAVR”). Next, as illustrated in FIG. 9C, upon completion of the procedure, and before withdrawing the vascular access sheath 110, a guide wire balloon device 120 (for example, any of the embodiments described elsewhere herein or in the applications incorporated by reference herein) may be inserted into the procedure sheath 110, such that a tip 121 of the guide wire device 120 is positioned past the sheath tip 111 inside the aorta 101 (or other body lumen).

Referring to FIGS. 9D and 9E, the sheath 110 may then be withdrawn, for example, under angiographic guidance, while maintaining the position of the guide wire device 120 in the iliofemoral artery 100. If sheath withdrawal uncovers a vascular injury, such as dissections 132 (shown in FIG. 9D) or perforations 134 (shown in FIG. 9E), expedient catheter management of the injury is possible by the guide wire device 120, which is positioned in the true lumen of the vessel 100. As shown in FIG. 9F, as a first step, the balloon 122 may be positioned at the location of the vascular injury 132 and inflated, in an effort to stabilize the vessel wall at the site of injury, and/or to bridge the complication for further treatment options.

With reference to FIG. 9G, the guide wire device 120 may provide a path for ipsilateral insertion of a treatment device, such as a catheter 134 with a balloon 136 and possibly a stent mounted on the balloon 136, for treating the vascular injury 132. In most or all embodiments, the guide wire device 120 may be “hubless,” meaning that once an inflation device (not shown) is removed from the device 120, one or more instruments may be passed over the proximal end of the guide wire device 120 without having to remove or navigate over a proximal hub. This hubless feature provides a significant advantage in ease of use for passing one or more additional devices to the area of the vascular injury. In other embodiments, alternative or additional treatment devices may be advanced over guide wire device 120, such as but not limited to any suitable catheter device, such as balloon expandable devices, stent delivery devices, graft delivery devices, radiofrequency or other energy delivery devices or the like. Under such scenarios, the device(s) 134 may be inserted into the target vessel over the guide wire device 120 while the injury is stabilized and bleeding is minimized by the expanded balloon 122, as shown in FIG. 9G.

Referring now to FIG. 9H, to facilitate positioning of a treatment device 134, the balloon 122 of the guide wire device 120 may be deflated and moved as desired within the vessel, for example, to an upstream location, as shown. Optionally, the tip 121 may be positioned past the iliofemoral segment 100 in the aorta 101 at any time during the procedure, for example, in order to prevent tip-related injury. In such procedures, the floppy tip 121, which may include the entire length distal to the balloon 122, may be sufficiently long to extend into the aorta when the balloon 122 is positioned in the iliofemoral segment 100. For example, in various embodiments, the tip 121 may be at least longer than the average length of the iliofemoral segment 100, such as at least about 15 cm, more preferably at least about 20 cm, and even more preferably between about 20 cm and about 25 cm.

The guide wire device 120 and therapeutic device(s) 134 are advanced to the injury site through vasculature on the same side of the patient's body that the procedural vascular access sheath 110 was placed. For the purposes of this application, this side of the patient is referred to as the ipsilateral side of a patient. In other words, in this application, “ipsilateral” refers to the side of the patient's body on which the main access was achieved for performing a given endovascular procedure. For example, the “ipsilateral femoral artery” or “ipsilateral iliofemoral artery” will generally be the artery in which a vascular access sheath 110 (or any other access device) is placed for advancing instruments to perform the intravascular procedure (TAVI, EVAR, etc.). “Contralateral” refers to the opposite side of the patient, relative to the procedure access side. In this regard, “ipsilateral” and “contralateral” relate to the side on which access is gained to perform the main procedure and do not relate to where the physician stands to perform the procedure. In any case, various embodiments of the methods and devices described herein may be used exclusively via an ipsilateral approach, exclusively via a contralateral approach, or interchangeably via an ipsilateral or contralateral approach.

The method just described in relation to FIGS. 9A-9I may have a number of advantages over the prior art contralateral balloon occlusion technique (CBOT). One advantage, for example, is that the guide wire balloon device 120 will typically be located very close to the vascular injury 132, 134 when the vascular sheath 110 is withdrawn. Thus, the balloon 122 may be inflated quickly within the iliofemoral artery 100, aorta 101, or femoral artery 102, perhaps after minor positional adjustments, to quickly occlude the vessel and stabilize the injury 132, 134 while treatment options are being assessed and prepared. Another potential advantage of the method described above is that only one combined guide wire balloon device 120 is needed to stop blood flow/stabilize the injury 132, 134 and to provide a path along which treatment device(s) 134 may be advanced into the vessel. In other words, the method does not require multiple different guidewires, guide catheters, introducer sheaths and the like, nor does it require difficult threading of a guidewire into a contralaterally placed sheath. In general, therefore, the described method may be easier and quicker to perform, thus facilitating a quicker and more effective vascular repair.

FIG. 10 illustrates an exemplary guide wire balloon system 200 (or “guide wire system”) for providing blood vessel occlusion, blood vessel injury stabilization and/or a device along which one or more treatment devices may be introduced during or after a large bore or other intravascular procedure may include a guide wire device 202 (or “guide wire balloon device”) and an inflation device 222. Optionally, the system 200 may also include an inflation medium container/injection device (not shown), such as but not limited to a syringe, a pump or the like. The guide wire device 202 extends from a hubless proximal end 205 to a distal end 219 and includes an expandable member such as an inflatable balloon 220 closer to the distal end 219 than the proximal end 205. The guide wire device 202 may be described as having a valve portion 204 (or “proximal portion”), a middle portion 210, a balloon portion 212 (or “transition portion”, “transition section” or “transition zone”) and a flexible tip 216 (or “J-tip,” “distal tip” or “distal portion”). These designations of the various portions of the guide wire device 202 are made for descriptive purposes only and do not necessarily connote specific demarcations or mechanical differences between the various portions, although in some embodiments, the various portions may have one or more unique characteristics.

The guide wire device 202 may further include a shaft 206 that extends from the valve portion 204 of the guide wire device 202 to at least a proximal end of the balloon 220. In one embodiment, the shaft 206 may be a hypotube, made of Nitinol, stainless steel, or some other metal, and may include a spiral cut 211 along part of its length to increase flexibility, as will be described in greater detail below. Inside the shaft 206, within the valve portion 204, there may reside an inflation hypotube 207 (or “inner tube”) with an inflation port 209, through which inflation fluid may be introduced. A valve cap 203 may be slidably disposed over the proximal end of the inflation hypotube 207, such that it may be moved proximally and distally to close and open, respectively, the inflation port 209. As best seen in the bottom magnified view of FIG. 10, a core wire 208 may be disposed within the shaft 206 along at least part of the middle portion 210 and may extend through the balloon portion 212 and in some embodiments through at least part of the distal tip portion 216. A coil 214 may be wrapped around part of the core wire 208 and may also extend beyond the core wire 208 to the extreme distal end 219. Various aspects and features of the shaft 206, inflation hypotube 207, core wire 208, coil 214, etc. will be described in further detail below.

The inflation device 222, which is also described in more detail below, may generally include a handle 224, a wire lumen 226 for inserting the guide wire device 202, and a locking inflation port 228. The handle 224 may be movable from a first position in which the guide wire device 202 may be inserted into the lumen 226 to a second position in which the handle 224 locks onto the shaft 206 and the valve cap 203. The handle may also be moveable from a valve-open position, in which inflation fluid may be passed into the inflation port 209 of the guide wire device 202, to a valve-closed position, in which the inflation fluid is trapped inside the balloon 220 and guide wire device 202. These positions and other aspects of a method for using the inflation device 222 will be described further below.

In one embodiment, the guide wire device 202 may have varying amounts of stiffness along its length, typically being stiffest at the proximal end 205 and most flexible at the distal end 219. The proximal/valve portion 204 and a proximal portion of the middle portion 210 of the guide wire device 202 are typically the stiffest portions of the device and will have sufficient stiffness to allow the device 202 to be advanced through a sheath and into a blood vessel, typically against the direction of blood flow (i.e., retrograde advancement). Along the middle portion 210, the device 202 may be relatively stiff at a most proximal end and quite flexible at a distal end (within, or adjacent the proximal end of, the balloon 220). This change in stiffness/flexibility may be achieved using any of a number of suitable mechanical means. In the embodiment shown, for example, the shaft 206 includes a spiral cut 211 along its length, where the spacing between the cuts becomes gradually less along the middle portion 210 from proximal to distal. In other words, the “threads” of the spiral cut are closer together distally. In alternative embodiments, increasing flexibility of the shaft 206 from proximal to distal may be achieved by other means, such gradually thinning the wall thickness of the shaft, using different materials along the length of the shaft or the like.

In the embodiment of FIG. 10, the spiral cut 211 may be configured such that the shaft 206 has a relatively constant stiffness along the valve portion 204 and a proximal part of the middle portion 210. As the shaft 206 approaches the proximal end of the balloon 220, the stiffness may fall off abruptly. In other words, the stiff shaft 206 has a significant drop-off in stiffness immediately proximal to the balloon 220. This type of stiffness/flexibility profile is in direct contrast to the typical prior art balloon catheter, which simply becomes more flexible at a gradual, consistent rate over its length. The unique stiffness profile of the guide wire device 202 may be advantageous, because maintaining significant stiffness along most of a proximal length of the device 202 provides for enhanced pushability against blood flow, while a significantly more flexible portion immediately proximal to, within, and distal to the balloon 220 will help to prevent injury to the vessel through which the device 202 is being advanced. A stiffer proximal portion 204 and middle portion 210 may also help temporarily straighten out a tortuous blood vessel, which may facilitate stabilizing and/or treating an injury in the vessel.

The top portion of FIG. 10 is a close up of the balloon section 212 of the guide wire device 202, with the balloon 220 removed. In this embodiment, the shaft 206 extends into a portion of the balloon section 212, with the spiral cut getting tighter, and then ends, leaving a small portion of the core wire 208 exposed. Inflation fluid exits from the distal end of the shaft 206 to inflate the balloon 220. The shaft 206 thus forms an inflation lumen (not visible in FIG. 15), and in the embodiment with the spiral cut 211, a coating or sleeve may be used to seal the shaft 206 to prevent inflation fluid from escaping the shaft 206 through the spiral cut 211. For example, a polymeric coating may be used, such as a shrink wrap coating, sprayed-on coating, dip coating, or the like. In alternative embodiments, the shaft 206 may end at the proximal end of the balloon 206 or may continue through the entire length of the balloon 220 and include one or more inflation ports in its sidewall. A distal portion of the core wire 208 is wrapped by the core wire 214. In these or other alternative embodiments, core wire 214 may stop at a distal end of the balloon 220 or alternatively extend all the way through the balloon 220. A number of various embodiments of the balloon section 212 will be described below in greater detail.

Referring now to the bottom close-up of FIG. 10, the core wire 208 may, in some embodiments, have a varying diameter at one or more points along its length. In alternative embodiments, it may have a continuous diameter. In the embodiment shown, for example, the core wire 208 has a relatively small diameter proximally, widens to a wider diameter, widens again to a widest diameter, and contracts gradually to a smallest diameter the flexible, J-tip portion 216. As will be described in greater detail below, the proximal end of the core wire 208 (not visible in FIG. 10) may also be widened, flattened or otherwise shaped to facilitate attaching the proximal end to an inner wall of the shaft 206 via gluing, welding, soldering or the like. The widest diameter section of the core wire 208, in this embodiment, is located where the distal end of the balloon 220 is mounted onto the core wire 208. This widest portion thus helps provide strength at an area of stress of the device 202. In some embodiments, the proximal end of the core wire 208 is attached to an inner surface of the shaft 206 by any suitable means, such as by welding, soldering, gluing, or the like. In some embodiments, the attachment point of the core wire 208 to the shaft 206 is proximal to the area along the shaft 206 where the spiral cut 211 begins. Alternatively, the core wire 208 may be attached at any other suitable location.

As illustrated in the bottom close-up of FIG. 10, in one embodiment, the diameter of the core wire 208 gets smaller and smaller distally along the length of the flexible J-tip portion 216, thus forming the most flexible, J-curved, distal portion of the guide wire device 202. In alternative embodiments, the core wire 208 may end proximal to the extreme distal end 219 of the guide wire device 202, and the coil 214 may continue to the distal end 219. In other alternative embodiments, the distal tip 216 may be straight, may include two core wires 208, may include more than two core wires 208, may be straightenable and/or the like. In the embodiment shown, the core wire includes a flat portion through the curve of the J-shape of the tip 216 and is attached to the coil 214 at the distal end 219 via a weld (or “weld ball”). The distal, curved portion of the J-tip is designed to be atraumatic to blood vessels through which it is advanced, due to its flexibility and shape.

The distal J-tip 216 of the guide wire device 202 may include special properties and/or features allowing for retrograde (against blood flow) insertion, maneuvering, and/or placement. For example, the “J-tip” shape of the distal tip 216 allows it to be advanced against blood flow without accidentally advancing into and damaging an arterial wall. Additionally, the distal tip 216 has a proximal portion through which the core wire 208 extends and a distal portion that is more flexible and includes only the coil 214. This provides for a slightly stiffer (though still relatively flexible) proximal portion of distal tip 216 and a more flexible (or “floppy”) distal portion of distal tip 216, thus providing sufficient pushability while remaining atraumatic. The extreme distal end 219 may also have a blunt, atraumatic configuration, as shown. In various embodiments, the distal tip 216 may also include a tip configuration, flexibility, radiopacity, rail support, core material, coating, and/or extension characteristics that enhance its function. Alternatively or in addition, device length considerations and/or overall shaft stiffness may be modified accordingly.

The core wire 208, the shaft 206 and the coil 214 may be made of any of a number of suitable materials, including but not limited to stainless steel, Nitinol, other metals and/or polymers. Each of these components may also have any suitable size and dimensions. For example, in one embodiment, the shaft 206 has an outer diameter of approximately 0.035 inches (approximately 0.9 mm). The guide wire device 202 may also have any suitable overall length as well as lengths of its various parts. Generally, the distal tip 216 will have a length that allows it to extend into an aorta when the balloon is inflated anywhere within an iliofemoral artery. In other words, the distal tip 216 may be at least approximately as long as the average iliofemoral artery. In various embodiments, for example, the distal tip 216 (measured from the distal end 219 of the device 202 to a distal end of the balloon 220) may be at least about 15 cm long, and more preferably at least about 20 cm long, and even more preferably between about 20 cm and about 25 cm long, or in one embodiment about 23 cm long. In various embodiments, the balloon section 212 of the device 202 may have a length of between about 10 mm and about 15 mm, or in one embodiment about 12 mm. In various embodiment, the middle section 210 of the device 202 may have a length of between about 70 cm and about 90 cm, and more preferably between about 75 cm and about 85 cm, or in one embodiment about 80 cm. And finally, in some embodiments, the valve section 204 may have a length of between about 10 cm and about 3 mm, or in one embodiment about 5 cm. Therefore, in some embodiments, the overall length of the device 202 might be between about 85 cm and about 125 cm, and more preferably between about 95 and about 115 cm, and even more preferably between about 105 cm and about 110 cm. Of course, other lengths for the various sections and for the device 202 overall are possible. For example, in some embodiments, the distal tip 216 may be longer than 25 cm, and in various embodiments, the overall length of the guide wire device 202 may range from may be longer than 115 cm. It may be advantageous, however, for ease of use and handling, to give the guide wire device 202 an overall length that is shorter than most currently available catheter devices. For an ipsilateral approach, the device 202 should generally have a length such that it is possible for the proximal portion 204 to extend at least partially out of the patient with the balloon 220 positioned within the iliofemoral artery and the distal end 219 residing in the aorta.

The balloon 220 of the guide wire balloon device 202 is generally a compliant balloon made of any suitable polymeric material, such as polyethylene terephthalate (PET), nylon, polytetrafluoroethylene (PTFE) or the like. The balloon 220 may be inflatable to any suitable diameter outside and inside the body. In one embodiment, for example, the balloon 220 may be inflatable within a blood vessel to a diameter of between about 6 mm and about 12 mm. In alternative embodiments, the balloon 220 may be semi-compliant or noncompliant. In some embodiments, the balloon 220 and/or portions of the device 202 immediately proximal and distal to the balloon 220 may include one or more radiopaque markers, to facilitate visualization of the balloon outside a patient's body using radiographic imaging techniques and thus facilitate placement of the balloon 220 in a desired location. The balloon 220 may be inflated, according to various embodiments, by any suitable inflation fluid, such as but not limited to saline, contrast solution, water, and air.

With reference now to FIG. 11, the guide wire balloon device may include one or more of the features described in connection with FIG. 10. The balloon segment 522 may include a balloon 520, a shaft 526 having a spiral cut 527 along at least a portion of its length proximal to a proximal end of the balloon 520, a core wire 528 extending from the distal tip 536 and through the extension balloon segment 522 and attached to the shaft 526 proximally, and a coil 524 disposed over at least a portion of the core wire 528 distal to the balloon 520. The core wire 528 may include a thinner balloon section 528′ underlying the balloon 520 and a flattened proximal end 528″, which may facilitate attachment to the shaft 526 via welding, gluing, soldering, or the like. As in most or all embodiments, the shaft 526 forms an inflation lumen 530 for inflating the balloon 520. Due to the spiral cut 527, the shaft 526 will typically be coated or covered with a sheath, such as a polymeric coating or sheath, to prevent inflation fluid (air, saline, etc.) from leaking through spiral cut 527. The balloon 520 may be mounted to the shaft 526 proximally and to the core wire 528 distally via threads 534 and epoxy 532 or other form of adhesive.

Embodiments described herein include an access wire balloon catheter that is attachable to an extension wire. The extension wire connects to the extra-corporeal tip of the access wire balloon catheter via a connection mechanism on the extra-corporeal tip of the access wire catheter and a corresponding/mating mechanism on one end of the extension wire. The extension wire may be a simple guidewire with a connection mechanism at one end and typically will not include a lumen or other features that would make manipulation and/or manufacturing more complex. The extension wire may be made of Nitinol, stainless steel, or any other suitable material, and may be made via any suitable wire making process. Together, the access wire balloon catheter and the connected extension wire typically have a length of at least about 200 cm and in some embodiments between about 260 cm and about 350 cm. Thus, the embodiments described herein provide the convenience, ease of use and lower cost of manufacturing of a short access wire balloon catheter with the overall length of a guide device that is typically needed for over-the-wire catheter exchanges.

In the typical embodiment, the access wire balloon catheter includes an inflation valve at or near the extracorporeal tip, so that when the extension wire is attached, the inflation valve resides between a free end of the extension wire and the balloon end of the access wire balloon catheter. According to various alternative embodiments, the add-on extension wire may be connected to the extracorporeal tip of the access wire device through one or multiple connectors. The mechanism for connecting the access wire to the extension wire may be mechanical, physical, magnetic, electromagnetic, optical, energy-based, chemical, and/or any other type of suitable mechanism, according to various embodiments. In some embodiments, the connection may be reversible, while in alternative embodiments, the connection may be permanent. Generally, the connection between the access wire balloon catheter and the extension wire is configured such that connecting and/or disconnecting the access wire device to the extension wire will not impact the basic functions of the access wire device, such as the ability to maintain balloon inflation and balloon positioning within the artery during connecting and disconnecting.

In various alternative embodiments, the access wire balloon catheter and the extension wire may have a number of different dimensions. For example, as discussed above, the total length of the access wire balloon catheter and extension wire, when connected, will typically be at least about 200 cm and in some embodiments between about 260 cm and about 350 cm. In one embodiment, for example, the access wire balloon catheter may have a length of about 85 cm, and the extension wire may have a length of about 175 cm. Any suitable combination of lengths may be used, as long as the access wire balloon catheter is long enough to reach a target location in a blood vessel while the extra-corporeal tip remains outside the patient, and as long as the total length of the access wire balloon catheter and the extension is long enough to allow for exchange of one or more secondary catheters. Additionally, the outer diameter of the access wire balloon catheter and the outer diameter of the extension wire typically are the same. This is important for allowing for smooth catheter exchange over the combined access wire device and extension.

In use, the shorter access wire balloon catheter may be inserted into the target blood vessel, positioned in a desired location for occluding blood flow, and then anchored in the vessel by inflating the balloon on the catheter. When the access wire balloon catheter is thus positioned, an extension wire may be attached to it outside the patient's body, and a treatment device, such as a secondary/treatment catheter, may be advanced over the access wire balloon catheter (the “primary” catheter) to the site of blood vessel injury. Using the access wire balloon catheter as a guiding device and as a blood flow occluder, any number of subsequent treatment devices may be advanced to and from the injury site to help treat the injury. At the end of the vessel repair, the balloon of the access wire device may be deflated, and the extension wire and access wire balloon catheter may be removed from the blood vessel. In some embodiments, it may be possible to detach the extension wire from the access wire balloon catheter before removing the latter from the blood vessel, if desired. Thus, the access wire balloon catheter may be made relatively short (for example about 85-200 cm in some embodiments), thus allowing for easy maneuverability, quick inflation and deflation, low risk of kinking, and low cost of manufacturing. Using the extension wire, the total length of the catheter can be extended during the part of the repair procedure when devices are exchanged over the access wire.

In some scenarios, all of the steps in the preceding paragraph may be performed by the same person. In some scenarios, it may be desirable for two or more people to carry out the steps in the preceding paragraph. For example, a first person may position the access wire balloon catheter and inflate the balloon. The first person may direct a second person to attach the extension wire to the access wire balloon and/or advance the primary catheter over the access wire balloon. In this scenario, the first person and the second person act in concert to treat the patient.

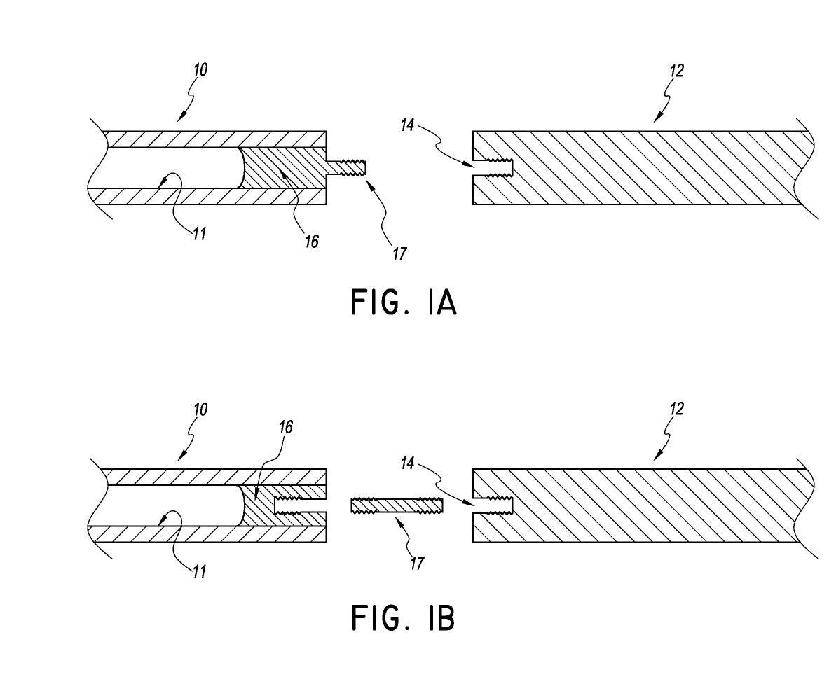

Referring now to FIGS. 1A and 1B, an extra-corporeal tip of one embodiment of an access wire balloon catheter 10 is illustrated, along with a mating end of an extension wire 12. In this embodiment, a threaded insert 16 resides within an inner wall 11 of access wire device 10. A threaded protrusion 17, which in some embodiments may be a rod, is used to connect the extension wire 12 via a corresponding threaded concavity 14. Insert 16 may be welded, attached with an adhesive, threadably locked, or otherwise connected to inner wall 11 of access wire device 10.

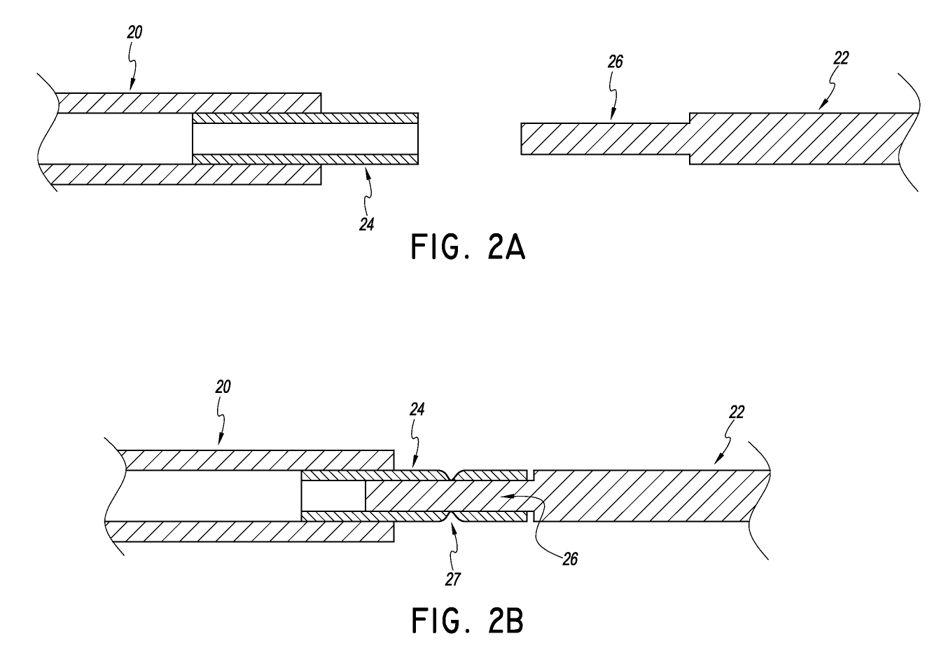

With reference to FIGS. 2A and 2B, in another embodiment, an extra-corporeal tip of an access wire balloon catheter 20 may include an attached tubular member 24. The tubular member 24 can be welded, swaged, or otherwise connected to an inner wall of the access wire balloon catheter. A protrusion 26 of an extension wire 22 may fit within tubular member 24, and tubular member 24 may be crimped with a crimping tool to form a deformation 27, thus attaching extension wire 22 to access wire catheter 20. This embodiment is an example of a permanent attachment between access wire device 20 and extension wire 22.

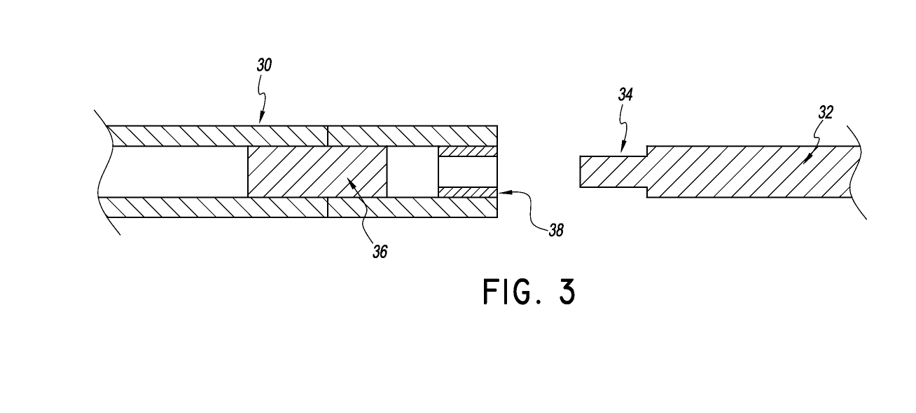

Referring now to FIG. 3, in an alternative embodiment, an extra-corporeal tip of an access wire balloon catheter 30 may include a friction fit material 38 and an insert 36. An extension wire 32 may include a protrusion 34, which fits inside friction fit material 38. In one embodiment, for example, friction fit material may be silicone. Insert 36 blocks inflation fluid (saline, air, etc.) from escaping through the end of extra-corporeal tip. The insert 36 may be welded or otherwise connected to an inner wall of the access balloon catheter 30.

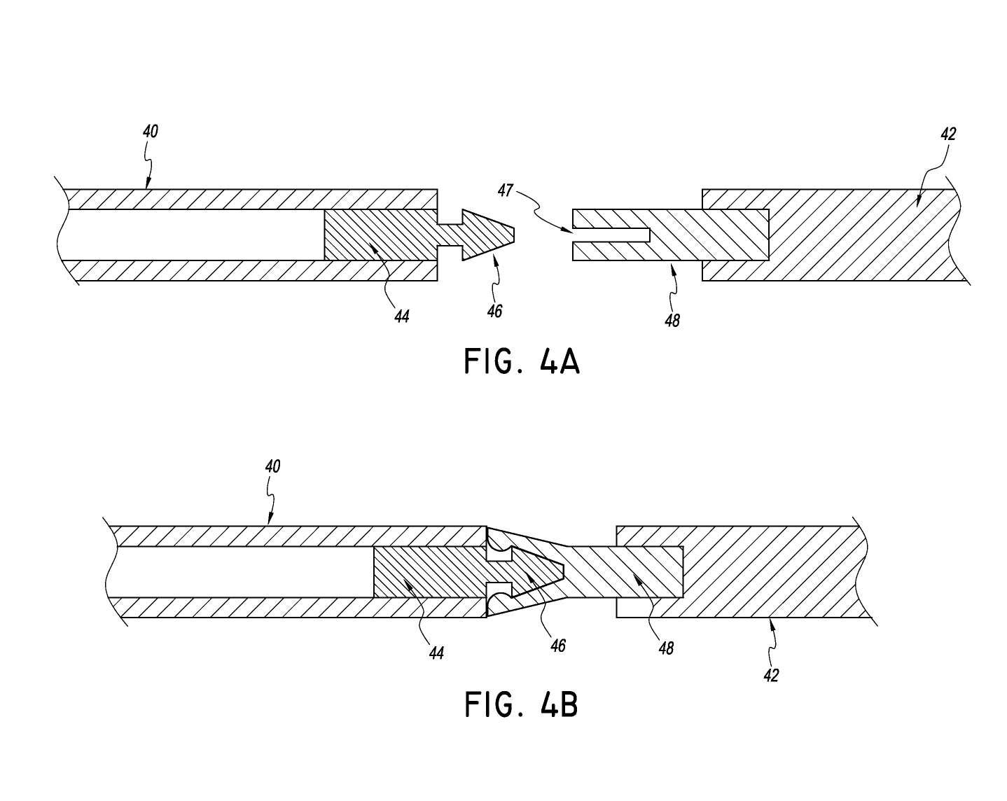

With reference now to FIGS. 4A and 4B, in another alternative embodiment, an extra-corporeal tip of an access wire balloon catheter 40 may include an insert 44 having a shaped protrusion 46, such as an arrow head shape as in the embodiment pictured. An extension wire 42 may include a protrusion 48 with a concavity 47 for accepting shaped protrusion 46. Protrusion 48 may be made of a conforming material, which is able to give and mold around shaped protrusion 46, as pictured in FIG. 4B. The insert can be welded, swaged, or otherwise connected to an inner wall of the access wire balloon catheter 40.

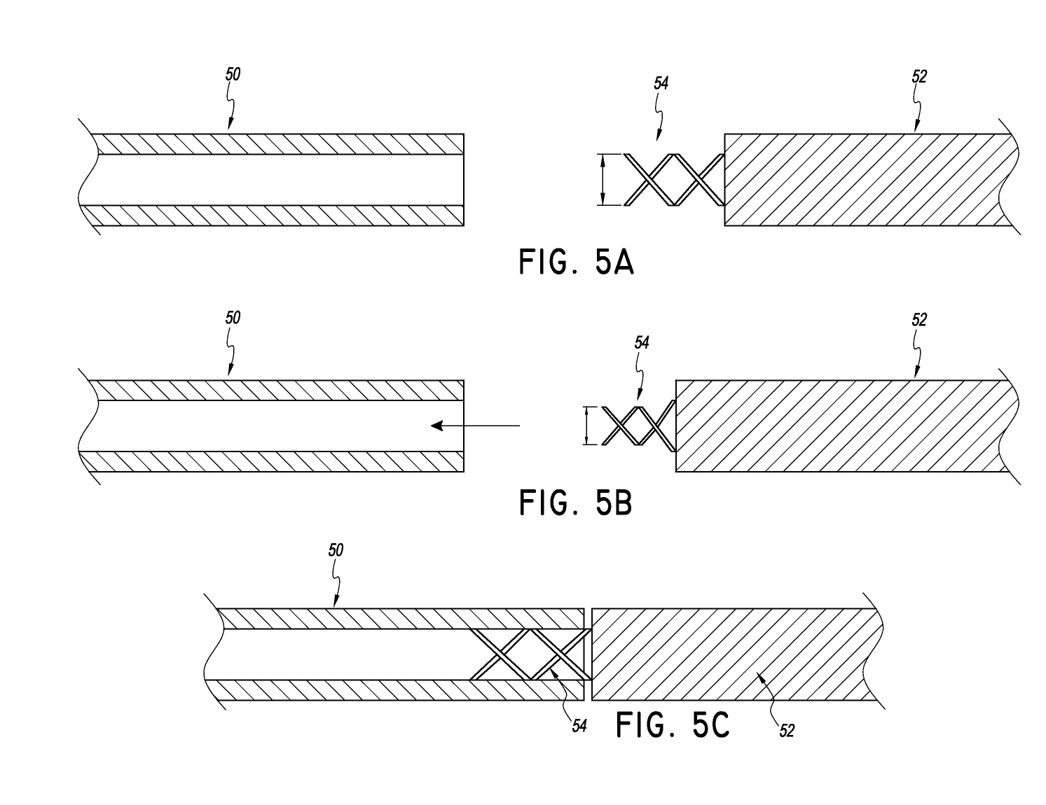

Referring now to FIGS. 5A-5C, in another embodiment, an extra-corporeal tip of an access wire balloon catheter 50 may connect with an extension wire 52 via a shape memory protrusion 54 on wire 52. In one embodiment, for example, protrusion 54 may have a default expanded state (FIG. 5A), may shrink when cooled (FIG. 5B) and may return to its default expanded state when allowed to return to room temperature (FIG. 5C). As illustrated in the figures, protrusion 54 may be inserted into the extracorporeal tip of access wire device 50 when in the cooled/smaller diameter configuration and then allowed to expand to form a connection via pressure fit. Protrusion 54 may have any suitable configuration, such as a mesh, lattice, or the like. The protrusion 54 can have a diameter of about 0.032 inches in the expanded state and a diameter of about 0.025 inches in the shrunken state.

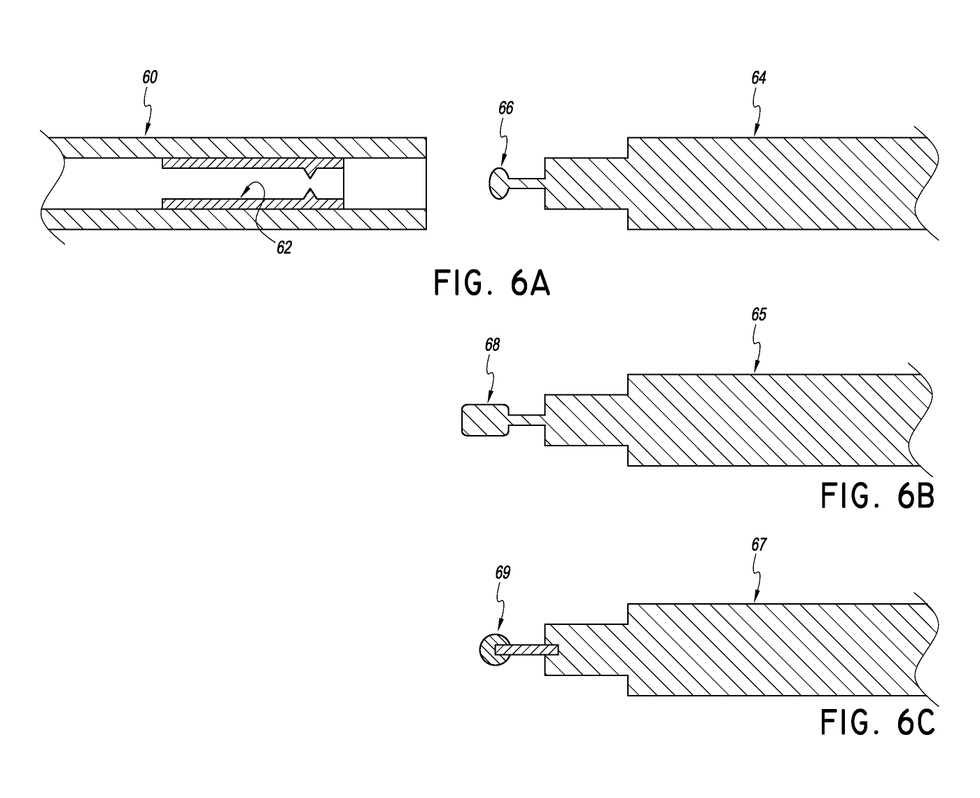

Referring to FIGS. 6A-6C, in another alternative embodiment, an extra-corporeal tip of an access wire balloon catheter 60 may include an insert 62 with a locking shape. In various embodiments, an extension wire 64, 65, or 67 may include a mating protrusion 66, 68, or 69, respectively, which fits into and locks with insert 62 of access wire catheter 60. In various embodiments, any suitable shape for insert 62 and protrusion 66, 68, 69 may be used. The insert 62 can be welded, swaged, or otherwise connected to an inner wall of the access wire balloon catheter 60.

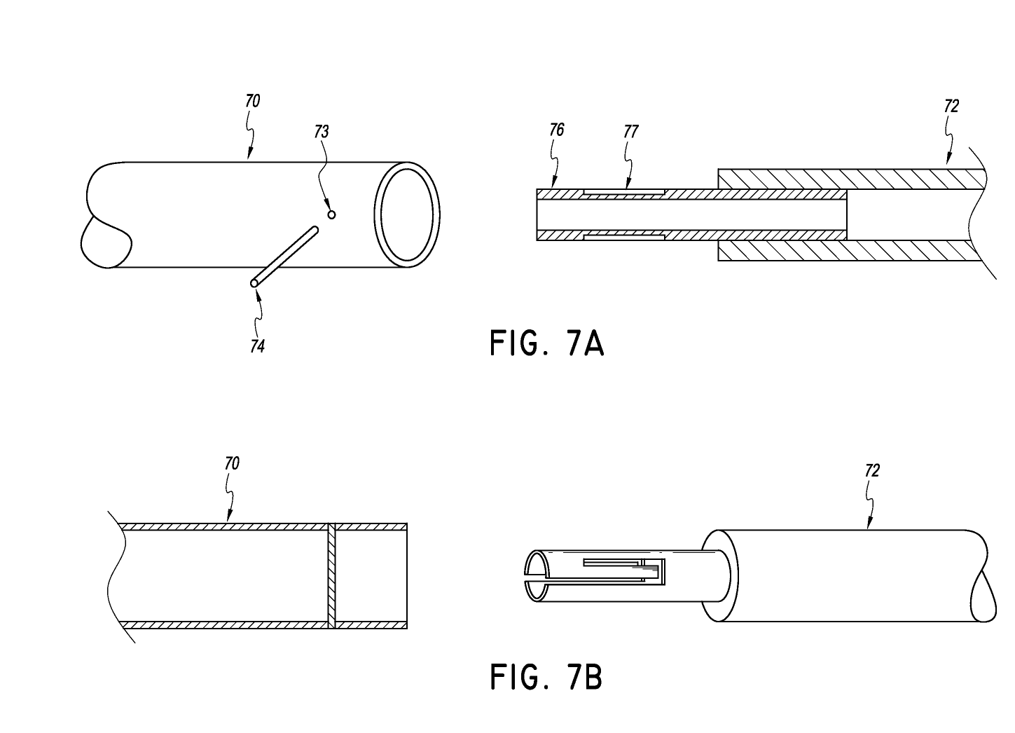

In another alternative embodiment, and with reference now to FIGS. 7A and 7B, an extra-corporeal tip of an access wire balloon catheter 70 may include an aperture 73 and a pin 74 (or “rod”) to fit through aperture 73. An extension wire 72 may include an insert protrusion 76, which includes a hook portion 77, which in some embodiments may be laser cut into protrusion 76. In use, protrusion 76 fits within the extra-corporeal end of access wire device 70 and is locked in place by inserting pin 74 into aperture 73. The pin 74 can form a press-fit with the balloon catheter 70 and/or extension wire 72. The pin can have a length about 0.035 inches.

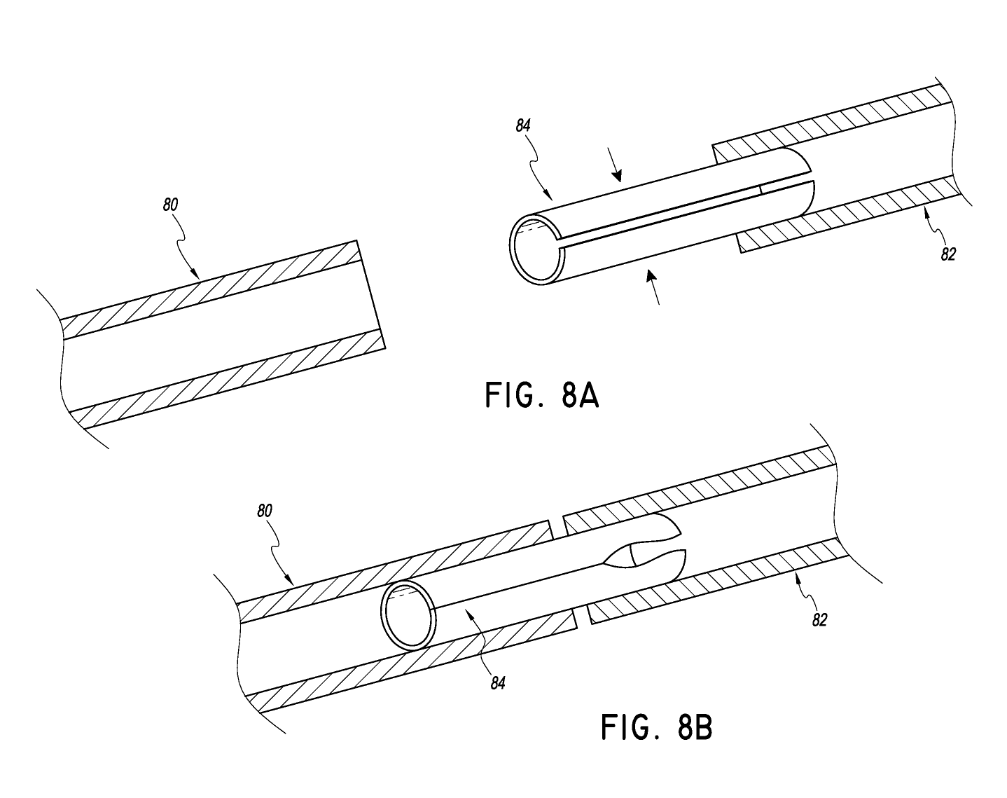

With reference to FIGS. 8A and 8B, in another alternative embodiment, an extracorporeal tip of an access wire balloon catheter 80 may be attached to an extension wire 82 using an insert 84 and interference fit. Insert 84 may be compressed via pressure during insertion into access wire balloon catheter 80 and/or extension wire 82, and it may then be allowed to expand after insertion to create the interference fit. In some embodiments, insert 84 may be welded to the inner wall of extension wire 82, so that it only connects to access wire device 80 via interference fit.

Elements or components shown with any embodiment herein are exemplary for the specific embodiment and may be used on or in combination with other embodiments disclosed herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

The terms “approximately,” “about,” and “substantially” as used herein represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount.

Some embodiments have been described in connection with the accompanying drawings. However, it should be understood that the figures are not drawn to scale. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various embodiments can be used in all other embodiments set forth herein. Additionally, it will be recognized that any methods described herein may be practiced using any device suitable for performing the recited steps.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives thereof.

Moreover, while illustrative embodiments have been described herein, the scope of any and all embodiments having equivalent elements, modifications, omissions, combinations (e.g., of aspects across various embodiments), adaptations and/or alterations as would be appreciated by those in the art based on the present disclosure. The limitations in the claims are to be interpreted broadly based on the language employed in the claims and not limited to the examples described in the present specification or during the prosecution of the application, which examples are to be construed as non-exclusive. Further, the actions of the disclosed processes and methods may be modified in any manner, including by reordering actions and/or inserting additional actions and/or deleting actions and/or performing the actions by a single actor or two or more actors in concert. It is intended, therefore, that the specification and examples be considered as illustrative only, with a true scope and spirit being indicated by the claims and their full scope of equivalents.

What is claimed is:

1. A method of treating an injured blood vessel of a patient, the method comprising:
inflating a balloon of an access wire balloon catheter within the injured blood vessel to reduce blood flow past an injury site in the vessel;
attaching an extension wire to an extra-corporeal end of the access wire balloon catheter that resides outside the patient, wherein, when the extension wire is attached, an inflation port of the access wire device is disposed outside the patient and between a free end of the extension wire and the balloon of the access wire balloon catheter;
advancing at least a first treatment catheter into the blood vessel over the access wire balloon catheter and at least a portion of the extension wire; and
treating the injured blood vessel using the first treatment catheter.

2. A method as in claim 1, further comprising:
removing the first treatment catheter from the blood vessel over the access wire balloon catheter and at least a portion of the extension wire;
advancing a second treatment catheter into the blood vessel over the access wire balloon catheter and at least a portion of the extension wire; and
further treating the injured blood vessel using the second treatment catheter.

3. A method as in claim 1, further comprising:
deflating the balloon; and
removing the access wire balloon catheter from the blood vessel while the extension wire is still attached.

4. A method as in claim 1, further comprising, before the inflating step:
detecting an injury in the injured blood vessel; and
positioning the balloon of the access wire balloon catheter device in a desired location in the blood vessel to provide at least partial occlusion of the vessel after inflation of the balloon.

5. A method as in claim 1, wherein inflating the balloon comprises inflating at a location of the vascular injury.

6. A method as in claim 1, wherein inflating the balloon comprises inflating at a location upstream of the vascular injury.

7. A method as in claim 1, wherein the first treatment catheter comprises a stent deployment catheter, and wherein treating the injury comprises placing the stent in the blood vessel.

8. A system for facilitating treatment of an injured blood vessel of a patient, the system comprising:
an access wire balloon catheter, comprising:
an elongate tubular body with a proximal end, a distal end, and a lumen extending longitudinally through at least part of the body;
an inflatable balloon disposed on the elongate body closer to the distal end than to the proximal end and in communication with the lumen;
a valve at or near the proximal end of the elongate body configured to couple with an inflation device to allow for inflation and deflation of the balloon; and
a first coupling member at the proximal end; and
an extension wire having a second coupling member at one end, wherein the first and second coupling members are configured to attach to one another to connect the proximal end of the access wire balloon catheter with one end of the extension wire,
wherein an outer diameter of the access wire balloon catheter is approximately the same as an outer diameter of the extension wire, at least in an area around a connection between the access wire balloon catheter and the extension wire.

9. A system as in claim 8, wherein the first and second coupling members attach to one another via a mechanism selected from the group consisting of threads, crimping, friction fit, shaped fit, phase change material(s), ball and socket fit, hook and pin, magnetics, and interference fit.

10. A system as in claim 8, wherein the access wire balloon catheter has a length of between about 85 cm and about 150 cm, and wherein a total length of the combined access wire balloon catheter and extension wire is between about 200 cm and about 350 cm.

11. A system as in claim 8, wherein, when the extension wire is connected to the access wire balloon catheter, the valve resides between a connection of the first and second connection members and the balloon of the access wire balloon catheter.

12. A device for facilitating treatment of an injured blood vessel of a patient, the device comprising:
an extension wire having a coupling member at one end for coupling with a corresponding coupling member on an access wire balloon catheter device used to occlude blood flow in the injured blood vessel, wherein an outer diameter of the extension wire is approximately the same as an outer diameter of the access wire balloon catheter, at least in an area around a connection between the extension wire and the access wire balloon catheter.

13. A device as in claim 12, wherein the coupling member couples with the corresponding coupling member via a mechanism selected from the group consisting of threads, crimping, friction fit, shaped fit, phase change material(s), ball and socket fit, hook and pin, and interference fit.

14. A device as in claim 12, wherein the extension wire has a length of between about 100 cm and about 215 cm.

15. A device as in claim 12, wherein the extension wire connects to one end of the access wire balloon catheter such that a valve of the access wire balloon catheter is distal to the connection.
